(19) United States
(12) Patent Application Publication
(10) Pub. No.: US 2023/0413879 A1
(43) Pub. Date: Dec. 28, 2023

(54) FOOD COMPRISING ISOSTEVIOL AND USES THEREOF

(71) Applicant: ISOFIRMS APS, Aarhus N (DK)

(72) Inventors: Gunnar KVISTGAARD, Bredebro (DK); Randi Kvistgaard SØNDERGAARD, Hørning (DK)

(21) Appl. No.: 18/252,964

(22) PCT Filed: Nov. 8, 2021
(86) PCT No.: PCT/EP2021/080931

§ 371 Date: May 15, 2023

(30) Foreign Application Priority Data
Nov. 16, 2020 (DK) PA 2020 70758

(51) Int. Cl. A23L 29/00; A23L 33/175; A61K 31/192; A23K 20/111

(52) U.S. Cl. CPC A23L 29/035; A23L 33/175; A61K 31/192; A23K 20/111

(57) ABSTRACT

The present invention relates to compositions or food ingredients comprising Isosteviol, food comprising isosteviol, systems comprising the composition or food ingredient and uses thereof.

### TECHNICAL FIELD OF THE INVENTION

The present invention relates to compositions or food ingredients comprising isosteviol, food comprising the composition or food ingredient, systems comprising the composition or food ingredient and uses thereof. In particular, the present invention relates to uses of isosteviol for increasing the muscle to fat ratio and/or decreasing the food conversion ratio (FCR) and/or increasing the uptake of proteins and/or carbohydrates and/or free amino acids of animal.

### BACKGROUND OF THE INVENTION

Isosteviol may be obtained by chemical modification of steviol glycosides (SG) from the plant Stevia rebaudiana L. The steviol glycosides derived from leaves of said plant are approved for use as food additives and sweeteners in the EU. By removing the sugar molecules in SG, under acidic conditions, Isosteviol is formed. Isosteviol in pure form is almost tasteless with no sweet taste at all.

Isosteviol has not previously been used or recognized as a compound with a nutritional/physiological capacity within the food or feed industry. However, previous studies have tried to use steviol glycosides as stevia extracts in feed studies before, mostly as sweetener to improve the feed uptake with negative conclusion on stevia inducing beneficial effects on feed intake and growth performance. As such, the study of J. O. Atteh et al., J. Anim. Physiol. Nutr., 2008, 92, pp. 640-649 discloses that feeding Stevia leaves or stevioside orally to broiler chickens had no positive effect on the broilers. In addition, the same study concluded that using Stevia and stevioside in a diet for broilers significantly increased the abdominal fat content of the chicken.

WO 2009/071099 relates to the use of isosteviol, steviol, and related compounds for elevating the plasma HDL-cholesterol level, lowering the plasma triglyceride level and/or reducing the body weight of a subject.

WO2014/173418 discloses that Stevia extract in a human trial with exhaustive exercise increases glycogen re-synthesis in muscles depleted in glycogen and restores muscle mass by increasing the rate of protein synthesis. However, the document provides no data in relation to isosteviol.

Hence, an improved food uptake would be advantageous, and in particular a more efficient and/or reliable food which can improve protein uptake and utilization would be advantageous.

### SUMMARY OF THE INVENTION

When burning fat efficiently, animals (including humans) use carbohydrates. Breakdown of carbohydrates forms oxaloacetic acid. Oxaloacetic acid is needed to breakdown fat into fuels for the cells. In order to gain a more healthy and beneficial body composition in terms of muscle to fat ratio, carbohydrates, amino acids and proteins must be effectively taken up and utilized by the body in order to alter the meat/fat ratio and the muscle to fat mass.

The present invention is based on the realization that isosteviol is able to improve the uptake and utilization of proteins, carbohydrates and/or amino acids from a diet. In the example section, animal (pig) studies are provided showing:
Feed comprising isosteviol increases the growth rate of pigs (example 2, FIG. 3 and example 8, FIGS. 10-11).
The increased growth was more pronounced when additional protein, carbohydrates and free amino acids were supplied (compared to a control group not receiving isosteviol) (example 2, FIG. 2).
Addition of isosteviol to the feed results in a higher overall uptake of the feed (decreased feed conversion ratio (FCR) (Example 3, FIG. 4 and example 8, FIGS. 12-13).
Isosteviol prevents and/or treats diarrhea (example 4, FIG. 4).
Isosteviol increases the meat/muscle percentage and thus also muscle/fat ratio in subjects (example 5).
Isosteviol increases the turnover rates of pigs for the farmer (example 6).

The animal studies shown in the example section can easily be translated into a human context. There is a great need for food/feed compositions, which may increase growth rate, but not the least improve the rebuilding of muscles, such as by improving the muscle to fat ratio in a subject.

Also, there is a need for compositions which can improve the uptake of high amounts of protein, carbohydrates and free amino acids without resulting in diarrhea. Diarrhea is a known side effect by athletes and other high protein intake in humans and in animals (such as pigs) in the farming industry.

Thus, an object of the present invention relates to the provision of an improved food for human and/or animal consumption.

In particular, it is an object of the present invention to provide a food that solves the above-mentioned problems of the prior art with efficient uptake of high amounts of protein, amino acids and carbohydrates. It is well known that very high amounts of protein and/or amino acids in a diet may result in diarrhea.

Thus, a first aspect of the present invention relates to a composition or food ingredient comprising isosteviol.

Another aspect relates to a composition or food ingredient comprising isosteviol, wherein the composition or food ingredient comprises an amount of protein of at least 30 w %. As shown e.g. in example 2, an increased growth was more pronounced when additional protein, carbohydrates and free amino acids were supplied (compared to a control group not receiving isosteviol).

A further aspect relates to a composition or food ingredient comprising isosteviol, wherein the composition or food ingredient comprises an amount of carbohydrate of at least 70 w %, such as at least 80 w %, or such as at least 90%. As shown e.g. in example 2, an increased growth was more pronounced when additional protein, carbohydrates and free amino acids were supplied (compared to a control group not receiving isosteviol).

A second aspect of the present invention relates to a food comprising the composition or food ingredient as described herein.

A third aspect of the present invention relates to a use of the composition or food ingredient as described herein, or the food as described herein, in/as a food.

Further aspects relates to uses for
decreasing the food/feed conversion ratio (FCR);
increasing the uptake of proteins and/or carbohydrates and/or free amino acids;
increasing the growth rate;
increasing the muscle to fat ratio;
improving weight-loss; and/or
gaining muscles;
increase in muscle mass in elderly or others where this is relevant such as subjects in physiotherapy treatment after in injuries and the like;
Increase muscle endurance;
Increase muscle recovery after exercise;
Increase muscle recovery after damage such as strains and similar muscular damage; and
decreasing output of stool.

A further aspect of the present invention relates to the composition or food ingredient as described herein, or the food as described herein, for use as a medicament.

Specific uses are for use
in the prevention and/or treatment and/or alleviation of a muscle disease;
in the prevention and/or treatment and/or alleviation of a digestive; disorder, such as diarrhea; and/or
as a blood sugar lowering agent; and/or
in rehabilitation/Recovery of muscle damage and/or atrophia; and/or
in conditions where muscle mass or muscle endurance must be increased such as in recovery from injuries; and/or
In fatty liver disorder; and/or
In the reduction in blood sugar, such as in in non-diabetic human and animals, and/or such as in the treatment, prevention or alleviation of prediabetes or type 2 diabetes.

An even further aspect of the present invention relates to a process for manufacturing of a food as described herein, the process comprising the steps of:
I. providing the composition or food as described herein,
II. bringing the composition or food ingredient into contact with one or more additional food component, such as a standard food and/or a supplementary food (or just water),
III. optionally, adding the ingredient to a capsule, tablet, sachet, or pouch, chewing tablet/gum, injectabilia and forms made for buccal absorbtion.

An additional aspect of the present invention relates to a feeding/drinking system comprising:
I. the composition or food ingredient as described herein,
II. a supplementary food and/or a standard food; and
III. Optionally, instructions for manufacturing a food as described herein by the process as described herein.

### BRIEF DESCRIPTION OF THE FIGURES

FIG. 1 shows control group (CG) at index 100. Isosteviol and protein group (IPG) exceeding the growth of control group by 4.0%.

FIG. 2 shows control group+protein (CPG) at index 100. Isosteviol and protein group (IPG) exceeding the growth of control+protein group by 5.8%.

FIG. 3 shows control group (CG) at index 100. Isosteviol group (IG) exceeding the growth of control group by 0.8%.

FIG. 4 shows pictures from pigsty showing increasingly stool (manure) contamination in the CPG group compared to group with same protein and Isosteviol (IPG).

FIG. 5 shows the measured meat percentage in average of the groups tested.

FIG. 6 shows the groups meat average percentage. An increase of the IPG and the IG groups compared to control as baseline is seen.

FIG. 7 shows the improved turn over time for the IPG group vs. the Control group. Improvement corresponds to improved turn over time of 4.8%.

FIG. 8 shows yearly turnover rate in production (times per year). The IPG group had significantly faster finish rate. Corresponding to an increased turnover rate of group IPG compared to CG by around 4.8%

FIG. 9 shows the improved turn over time for the IG group vs. the Control group. Improvement corresponds to improved turn over time of 1%.

FIG. 10 shows improved weight gain for treatment 1 (+Isosteviol) versus negative control (−isosteviol). Until week 3: observed superiority of Treatment 1 versus negative control group. Week 3-6 and week 1-6: Observed relevant superiority, significant proven (p<0.0023).

FIG. 11 shows improved weight gain for treatment 2 (+Isosteviol) versus negative control (−Isosteviol). Until week 3: observed very small superiority of Treatment 2 versus negative control group. Week 3-6 and week 1-6: Observed relevant superiority, significant proven (p<0.0158).

FIG. 12 shows an improved Feed conversion ratio (FCR) for treatment 1 (+Isosteviol) versus negative control (−isosteviol). More than relevant superiority for treatment 1 versus the negative control group can be observed.

FIG. 13 shows an improved Feed conversion ratio (FCR) for treatment 2 (+Isosteviol) versus negative control (−isosteviol). More than relevant superiority for treatment 2 versus the negative control group can be observed.

The present invention will now be described in more detail in the following.

### DETAILED DESCRIPTION OF THE INVENTION

### Definitions

Prior to discussing the present invention in further details, the following terms and conventions will first be defined:

### Standard Food/Feed

In the present context, the term “standard food/feed” is to be understood as a food comprising the standard components making up a food/feed. Thus, a standard food/feed is to be understood to comprise at least:
Protein and/or free amino acids;
Carbohydrate;
Vitamins;
Minerals and
Optionally fat.

### Supplementary Food/Feed

In the present context the term “supplementary food/feed” is to be understood as an additional component which may be added to a food (such as a standard feed/food), to increase the amount of specific components in the mixture. Thus, a supplementary food/feed is to be understood to comprise at least:
Protein; and/or
Free amino acids; and/or
Carbohydrates; and/or
Optionally enzymes; and/or
Vitamins; and/or
Minerals; and/or
Fat.

Preferably, the supplementary food/feed comprises protein and/or free amino acids. It is also preferred that the supplementary food/feed comprises carbohydrates.

### Food

In the present context the term “food” or “food ingredient” is to be understood as also covering “feed” and “feed ingredient”. Thus, the food according to the invention may refer both to food/feed suitable for human and/or animal consumption and the term food ingredient according to the invention may refer both to food/feed suitable for human and/or animal consumption.

Generally speaking, the term “feed” refers to animal consumption and the term “food” refers to human or animal consumption. In the broad sense of the invention the terms may be used interchangeably.

### Steviol

In an embodiment, in the present context, the term “steviol” refers to the compound according to Formula (1)

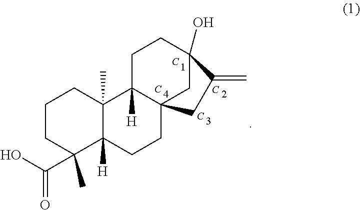

The structure of steviol is present in steviol glycosides and the compound may therefore be obtained from e.g. Stevia plants. Notice, that the bridge between C₁ and C₄ has a methylene group at C₂.

### Isosteviol

In a preferred embodiment, in the present context, the term “isosteviol” refers to the compound according to Formula (2):

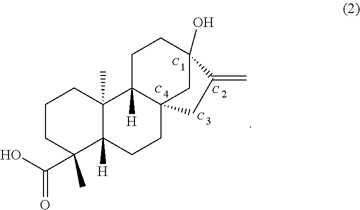

Isosteviol has an oxo group at C₂ and the bridge connecting C₁ and C₄ is below the plane of the six-membered ring unlike the structure of steviol wherein the bridge is above the ring.

### Steviol Glycosides

In the present context, the term “steviol glycosides” refers to a group of compounds wherein each member of the group is a glycoside of steviol. Steviol glycosides may be represented by Formula (3):

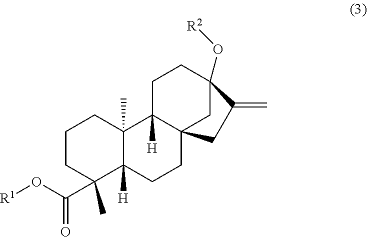

wherein, at least one of R¹ and R² is a sugar moiety (such as disaccharide or polysaccharide) bound by glycosidic bond to the anomeric carbon of the sugar and if not a saccharide, then R¹ or R² is a hydrogen atom.

As an example, the compound stevioside is the compound wherein R¹ is a monosaccharide moiety of beta-D-glucopyranosyl, and R² is a disaccharide moiety of 2-O-beta-D-glucopyranosyl-beta-D-glucopyranosyl.

Steviol glycosides are naturally present in specific plants. In particular, the steviol glycosides in S. rebaudiana leaves include: Stevioside, Rebaudioside A, Rebaudioside C, Dulcoside A, ebaudioside B, Rebaudioside D, Rebaudioside E. The last three are present only in minute quantities. A commercial steviol glycoside mixture extracted from the plant was found to have about 80% stevioside, 8% rebaudioside A, and 0.6% rebaudioside C. The Chinese plant Rubus chingii produces rubusoside, a steviol glycoside not found in Stevia.

### Composition or Food Ingredient

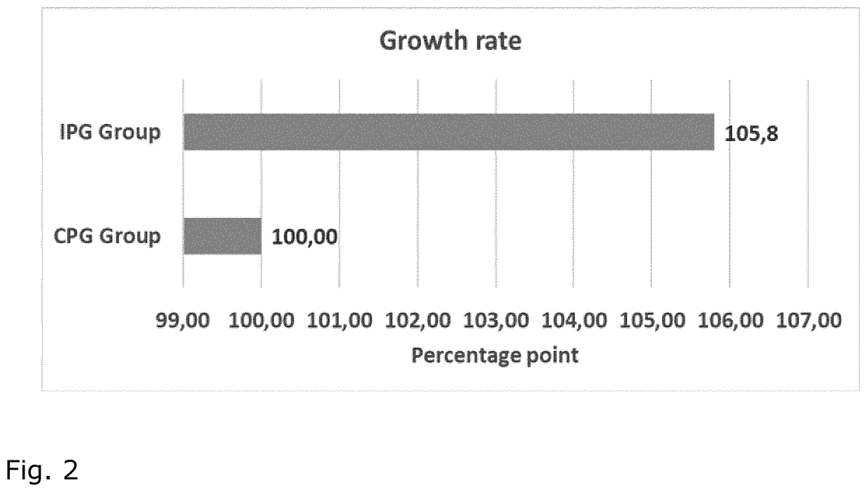

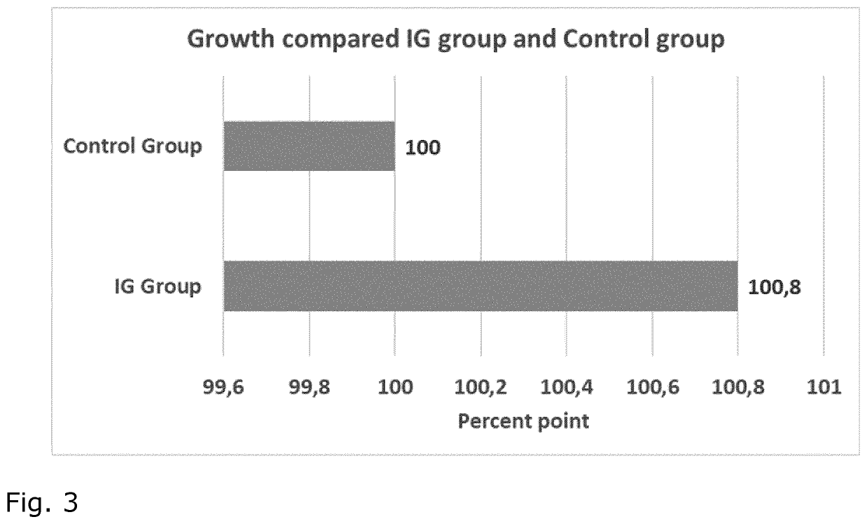

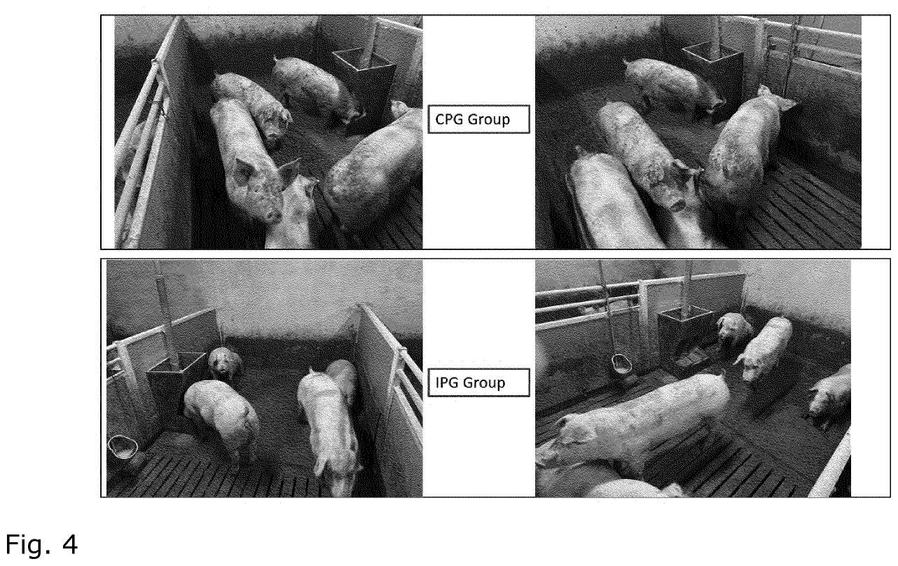

As also outlined above, the present invention is based on the realization that isosteviol is able to improve the uptake and utilization of proteins, carbohydrates and/or amino acids from a diet. In the example section, animal (pig) studies are provided showing:
Feed comprising isosteviol increases the growth rate of pigs (example 2, FIG. 3).
The increased growth was more pronounced when additional protein and free amino acids were supplied (compared to a control group not receiving isosteviol) (example 2, FIG. 2).
Addition of isosteviol to the feed results in a higher overall uptake of the feed (Example 3, FIG. 4).
Isosteviol prevents and/or treats diarrhea (example 4, FIG. 4).
Isosteviol increases the meat/muscle percentage and thus also muscle/fat ratio in subjects (example 5).
Isosteviol increases the turnover rates of pigs for the farmer (example 6).
Isosteviol decreases the amount of nutritional ingredients in stool (example 3).

Thus, a first aspect of the present invention relates to a composition or food ingredient comprising isosteviol. The composition or food ingredient according to the invention may be suitable to be a food product in its own right, but may also form part of a food product and thereby function as an ingredient in such food/feed product.

An embodiment of the present invention relates to the composition or food ingredient, having an amount of isosteviol of at least 0.1 w %, such as in the range of 0.1 w % to 100 w %, such as 10 w % to 99.99 w %, such as 20 w % to 99.99 w %, such as 30 w % to 99.99 w %, preferably such as 35 w % to 99.99 w %, more preferably such as 39.9 w % to 99.99 w %, based on the total weight of the composition or food ingredient. As shown in examples 4 and 7 the amount of isosteviol may vary greatly, when present in the composition or food ingredient according to the invention. A composition or ingredient may consist almost entirely of isosteviol or be mixed with other components.

Lower amounts of isosteviol may also be present in the composition or food ingredient according to the invention. Thus, in an embodiment, the composition or food ingredient has an amount of isosteviol of less than 50 w %, such as less than such as less than 30w %, such as less than 20w %, such as less than or such as less than 5w %. In another embodiment, the composition or food ingredient has an amount of isosteviol in the range of 0.1 w % to 50 w %, such as 0.1 w % to 40 w %, such as 0.5 w % to 30 w %, such as 0.5 w % to 20 w %, preferably such as 0.5 w % to 10 w %, more preferably such as 1 w % to 10 w %, based on the total weight of the composition or food ingredient. Example 7 shows different compositions, food ingredients and foods according to the invention. The specific amounts depends on the specific use but also allowable daily intake for the animal in question and the amounts given to the animal (such as pigs or humans).

In an embodiment, the composition or food ingredient is in powder form.

An embodiment of the present invention relates to the composition or food ingredient, wherein the amount of steviol glycosides make up 0 w % to 7 w % of the total weight of the composition or food ingredient, such as in the range 0-5%, such as in the range 0-3%, such as 0-2%, such as 0-1%, such as 0-0.5% or such as the amount of steviol glycosides make up 0-0.05% of the total weight of the composition or food ingredient, or such as the composition or food ingredient is substantially free from steviol glycosides. Stevia plants are considered to be free from isosteviol.

An embodiment of the present invention relates to the composition or food ingredient, wherein the isosteviol is isolated, purified, and/or synthetically produced.

An embodiment of the present invention relates to the composition or food ingredient, wherein the composition or food ingredient further comprises one or more of a source of protein.

An embodiment of the present invention relates to the composition or food ingredient, wherein the amount of protein is at least 0.1 w %, such as at least 1 w %, such as at least 5 w %, such as at least 10 w %, such as at least 20 w %, such as at least 30 w %, such as at least 40 w %.

In a related embodiment, the amount of protein is in the range of 1 w % to 99.9 w %, such as in the range of 10-95 w %, such as 15-95 w %, such as 15-80 w %, such as 15-70 w %, such as 20-60 w %, such as 20-55 w %, based on the total weight of the composition or food ingredient.

In yet a related embodiment, the amount of protein is in the range of 30 w % to 99.9 w %, such as in the range of 30-95 w %, such as 30-80 w %, such as 30-70 w %, such as 30-60 w %, such as 30-55 w %, based on the total weight of the composition or food ingredient. In an embodiment, the amount of protein is in the range 30-70 w %.

An embodiment of the present invention relates to the composition or food ingredient, wherein the source of protein is selected from the group consisting of alfalfa, cotton seed, fishmeal, meat, oilseed rape, oil palm, coconut, sesame, crambe, clover, bean, pea, chickpea, lentil, lupin, mesquite, carob, peanuts, tamarind, maize, quality protein maize, genetically improved protein crops, sunflower, hemp, insects, soya, and combinations thereof.

An embodiment of the present invention relates to the composition or food ingredient, further comprising one or more of a source of carbohydrate.

An embodiment of the present invention relates to the composition or food ingredient, wherein the amount of carbohydrate is at least 0.1 w %, such as in the range of 1 w % to 95 w %, such as in the range of 10-95 w %, such as 15-95 w %, such as 20-95 w %, such as 25-95 w %, such as 30-95 w %, such as 30-90 w %, such as 30-80 w %, such as 30-70 w %, such as 30-65 w %, based on the total weight of the composition or food ingredient.

In yet an embodiment, the composition or food ingredient comprises an amount of carbohydrate in the range 0.1-65w %

An embodiment of the present invention relates to the composition or food ingredient, wherein the source of carbohydrate is selected from the group consisting of wheat, barley, rye, corn, oat, sugar beet molasses and combinations thereof.

An embodiment of the present invention relates to the composition or food ingredient, wherein the source of carbohydrate comprises one or more carbohydrate selected from the group consisting of sugar, starch, and cellulose.

An embodiment of the present invention relates to the composition or food ingredient, further comprising one or more free amino acids.

An amino acid is a compound comprising at least one amino (—NH₂) group and at least one carboxylic acid (—COOH) group. A free amino acid is an amino acid molecule which is not bound or otherwise attached to any other compounds or moieties. A non-exclusive list of common free amino acids includes: histidine, isoleucine, leucine, lysine, methionine, phenylalanine, threonine, tryptophan, valine, arginine, cysteine, glutamine, glycine, proline, serine, tyrosine, taurine, alanine, asparagine, aspartic acid, and glutamic acid.

An embodiment of the present invention relates to the composition or food ingredient, wherein the amount of free amino acids is at least 0.1 w %, such as in the range of 0.1 w % to 95 w %, such as in the range of 1-80 w %, such as 1-70 w %, such as 1-60 w %, such as 5-60 w %, such as 10-60 w %, such as 10-50 w %, such as 10-45 w %, based on the total weight of the composition or food ingredient.

A preferred embodiment of the present invention relates to the composition or food ingredient, wherein the one or more free amino acids is selected from the group consisting of histidine, isoleucine, leucine, lysine, methionine, phenylalanine, threonine, tryptophan, valine, taurine and combinations thereof.

An embodiment of the present invention relates to the composition or food ingredient, further comprising taurine.

An embodiment of the present invention relates to the composition or food ingredient, further comprising one or more of a source of fat.

An embodiment of the present invention relates to the composition or food ingredient, wherein the source of fat is selected from the group consisting of plant oil, vegetable fat/oil, animal fat, liquid grease, and milk fat.

An embodiment of the present invention relates to the composition or food ingredient, wherein the source of fat comprises one or more lipid selected from the group consisting of monoglyceride, diglyceride, triglyceride, fatty acid, and phospholipid.

An embodiment of the present invention relates to the composition or food ingredient, wherein the amount of fat is at least 0.1 w %, such as in the range of w % to 60 w %, such as in the range of 0.1-50 w %, such as 0.1-40 w %, based on the total weight of the composition or food ingredient.

An embodiment of the present invention relates to the composition or food ingredient, further comprising crude fibre, and/or crude ash, and/or nutrient.

An embodiment of the present invention relates to the composition or food ingredient, wherein the nutrient is selected from the group consisting of calcium, sodium, phosphor, iron, iodine, selenium, cobber, zinc, manganese, E-vitamin, A-vitamin, D3-vitamin, and 6-phytase.

An embodiment of the present invention relates to the composition or food ingredient, wherein the composition or food ingredient is in the form of grains, pellets, liquid, solid, slurry or powder.

In an embodiment, Isosteviol in a composition or a food ingredient according to the present invention may be used in food products alone and/or in combination with other substances in the following food product categories:
Food supplements as per EU directive 2002/46 and amendments.
Food for specific groups as per EU regulation (EU) No 609/2013 of the European Parliament and the Council on food intended for infants and young children, food for special medical purposes, and total diet replacement for weight control.
Sports nutrition products which are as is regulated under general EU food law. This is Products that can be defined as Sport food; means food specifically designed, formulated, and/or marketed in relation to physical activity, physical performance and/or post-exercise recovery. Thus, Sports food has a nutritional composition adapted for a physical activity (before, during and after). A range of specific information is provided to consumers to explain the correct use of sports foods, including: Nutritional characteristics of sports products; Condition of use whether before, during, or after exercise; and recommendation of daily intake or intake during training, which helps to avoid overdose and provide the right amount of nutrients before, during or after exercise.

### Food Comprising Isosteviol

A second aspect of the present invention relates to a food comprising the composition or food ingredient as described herein.

In a related aspect, the invention relates to a food comprising or consisting of the composition or food ingredient according to the invention, wherein the food comprises an amount of protein of at least 30 w %.

In an embodiment, the food comprises an amount of isosteviol of at least 0.00001 w %, such as at least 0.0001, such as at least 0.001 w %, such as at least 0.01, such as at least 0.1 w %, such as at least 1 based on the total weight of the food.

In another embodiment, the food comprises at the most an amount of isosteviol of 20 w %, such as at the most 17 w %, such as at the most 13 w %, such as at the most 10 w %, such as at the most 8 w %, such as at the most 5 w %, such as at the most 3 w % based on the total weight of the food.

An embodiment of the present invention relates to the food, wherein the food comprises an amount of isosteviol in the range of 0.00001 w % to 20 w %, such as 0.00001 w % to 17 w %, such as 0.00001 w % to 13 w %, such as 0.00001 w % to 10 w %, such as 0.00001 w % to 8 w %, preferably such as 0.00001 w % to 5 w %, more preferably such as 0.00001 w % to 3 w % based on the total weight of the food. In another embodiment, the food comprises an amount of isosteviol in the range of 0.00001 w % to 20 w %, such as 0.0001 w % to 17 w %, such as 0.001 w % to 13 w %,such as 0.01 w % to 10 w %, such as 0.1 w % to 8 w %, preferably such as 1 w % to 5 w %, more preferably such as 1 w % to 3 w % based on the total weight of the food. Again, final amounts depends on e.g. type of food, allowable daily intake and the amount given to an animal per day.

An embodiment of the present invention relates to the food, wherein the food comprises an amount of isosteviol in the range of 0.0001 g/kg to 200 g/kg, such as 0.0001 g/kg to 170 g/kg, such as 0.0001 g/kg to 130 g/kg, such as 0.0001 g/kg to 100 g/kg, such as 0.0001 g/kg to 80 g/kg, preferably such as 0.0001 g/kg to 50 g/kg, more preferably such as 0.0001 g/kg to 30 g/kg based on the total weight of the food.

An embodiment of the present invention relates to the food, wherein the amount of protein is in the range of 0 w % to 99.99999 w %, such as in the range of 1 w % to 99.9 w %, such as in the range of 2-75 w %, such as 2-55 w %, such as 2-45 w %, such as 2-40 w %, such as 2-35 w %, based on the total weight of the food.

In yet an embodiment, the amount of protein is at least 20%, such as in the range of 20 w % to 99.9 w %, such as in the range of 30-90 w %, such as 40-90 w %, such as 50-99 w %, such as 60-99 w %, or such as 65-99 w %, based on the total weight of the food. For example, a protein powder supplemented with isosteviol will comprise high amounts of protein.

An embodiment of the present invention relates to the food, wherein the amount of carbohydrate is in the range of 0 w % to 99.99999 w %, such as in the range of 1 w % to 95 w %, such as in the range of 5-95 w %, such as 10-95 w %, such as w %, such as 15-90 w %, such as 15-85 w %, such as 10-80 w %, such as w %, such as 20-70 w %, such as 25-65 w %, based on the total weight of the food. In an embodiment, the amount of carbohydrate is in the range 25-65 w %, based on the total weight of the food.

An embodiment of the present invention relates to the food, wherein the amount of fat is in the range of 0 w % to 99.99999 w %, such as in the range of 0.1 w % to 60 w %, such as in the range of 0.3-60 w %, such as 0.5-60 w %, such as 1-60 w %, such as 1-50 w %, such as 1-40 w %, such as 1-30 w %, such as 1-20 w %, such as 1-15 w %, based on the total weight of the food. In an embodiment, the amount of fat is in the range of 0.1 w % to 60 w %, based on the total weight of the food.

An embodiment of the present invention relates to the food, wherein the amount of free amino acid is in the range of 0 w % to 99.99999 w %, such as in the range of 0.1 w % to 95 w %, such as in the range of 0.1-75 w %, such as 0.1-65 w %, such as 0.1-55 w %, such as 0.3-55 w %, such as 0.5-55 w %, such as 1-55 w %, such as 1-45 w %, such as 1-35 w %, such as 1-25 w %, based on the total weight of the food. In an embodiment, the amount of free amino acid is in the range 0.1-65 w %, such as 0.1-55 w %, such as 0.3-55 w %, such as 0.5-55 w %, such as 1-55 w %, such as 1-45 w %, such as 1-35 w %, such as 1-25 w %, based on the total weight of the food.

A food composition considered particularly useful for feeding pigs/sows conforms to an embodiment of the present invention relating to the food, wherein the food comprises:
i) 3 w % to 30 w % of protein,
ii) 50 w % to 95 w % of carbohydrate,
iii) 0.3 w % to 10 w % of free amino acids,
iv) 1 w % to 8 w % of fat,
v) 0.00001 w % to 5 w %isosteviol, and
   wherein, each w % is calculated based on the total weight of the food.

An embodiment directed to a food considered particularly useful for feeding grower/slaughter pigs (pigs of approximately 30 kg-125 kg bodyweight) of the present invention relates to the food, wherein the food comprises:
i) 1 w % to 20 w % of protein,
ii) 15 w % to 40 w % of carbohydrate,
iii) 0.3 w % to 10 w % of free amino acids,
iv) 0.5 w % to 10 w % of fat,
v) 0.00001 w % to 5 w % isosteviol, and
   wherein, each w %is calculated based on the total weight of the food.

Food considered especially useful for feeding piglets (pigs of less than 35 kg bodyweight) is described by an embodiment of the present invention relating to the food, wherein the food comprises:
i) 3 w % to 33 w % of protein,
ii) 50 w % to 95 w % of carbohydrate,
iii) 0.3 w % to 10 w % of free amino acids,
iv) 1 w % to 8 w % of fat,
v) 0.00001 w % to 5 w %isosteviol, and
   wherein, each w %is calculated based on the total weight of the food.

Pigs and piglets may be fed with a mixture of food/food ingredient and/or a supplementary feed having a composition according to an embodiment of the present invention relating to the food, wherein the food comprises:
i) 22 w % to 65 w % of protein,
ii) 3 w % to 20 w % of carbohydrate,
iii) 25 w % to 65 w % of free amino acids,
iv) 2 w % to 12 w % of fat,
v) 0.00001 w % to 5 w %isosteviol, and
   wherein, each w %is calculated based on the total weight of the food.

A food composition considered particularly useful for feeding fish conforms to an embodiment of the present invention relating to the food, wherein the food comprises:
i) 25 w % to 65 w % of protein,
ii) 5 w % to 35 w % of carbohydrate,
iii) 10 w % to 55 w % of fat,
iv) 0.00001 w % to 5 w %isosteviol, and
   wherein, each w %is calculated based on the total weight of the food.

An embodiment directed to a food considered particularly useful for feeding racehorses and other high performing animals according to the present invention relates to the food, wherein the food comprises:
i) 2 w % to 22 w % of protein,
ii) 15 w % to 95 w % of carbohydrate,
iii) 2 w % to 10 w % of fat,
iv) 0.00001 w % to 5 w %isosteviol, and
   wherein, each w %is calculated based on the total weight of the food.

Working animals which are subject to extensive exercise are considered to require a food according to an embodiment of the present invention relating to the food, wherein the food comprises:
i) 2 w % to 42 w % of protein,
ii) 15 w % to 95 w % of carbohydrate,
iii) 2 w % to 25 w % of fat,
iv) 0.00001 w % to 8 w %isosteviol, and
   wherein, each w %is calculated based on the total weight of the food.

Food, such as a protein powder is considered to be a composition according to an embodiment of the present invention relating to the food, wherein the food comprises per 100 g: fat 0-5% protein components incl. free amino acids 10-95% carbohydrates incl. sugars 0-20%, isosteviol 1-10%.

An embodiment of the present invention relates to the food, wherein the food comprises a drinkable liquid.

Food, such as a vitamin water or a sports water is considered to be a composition according to an embodiment of the present invention relating to the food, wherein the food comprises per 100 g: fat 0-2 w %, protein components incl. free amino acids 0-10 w %, carbohydrates incl. sugars 1-30 w %, and isosteviol 0.00001 w % to 5 w %.

Food, such as a sports gel is considered to be a composition according to an embodiment of the present invention relating to the food, wherein the food comprises per 100 g: fat 0-10 w %, protein components incl. free amino acids 0-10 w %, carbohydrates incl. sugars 10-95 w %, isosteviol 1-8 w %.

Food, such as a protein shake is considered to be a composition according to an embodiment of the present invention relating to the food, wherein the food comprises per 100 g: fat 0-10 w %, protein components incl. free amino acids 0-10 w %, carbohydrates incl. sugars 5-80 w %, isosteviol 1-8 w %.

Food, such as a supplement shake for elderly or a malnutritioned/undernutritioned people is considered to be a composition according to an embodiment of the present invention relating to the food, wherein the food comprises per 100 ml: fat 0-20 w %, protein components incl. free amino acids 5-30 w %, carbohydrates incl. sugars w5-80%, isosteviol w1-8%.

Food, such as a supplement fibre shake is considered to be a composition according to an embodiment of the present invention relating to the food, wherein the food comprises per 100 ml: fat 0-20 w %, protein components incl. free amino acids 3-30%, carbohydrates incl. sugars 5-80 w %, fibers 1-15 w %, isosteviol 1-8 w %.

Food, such as a food supplement is considered to be a composition according to an embodiment of the present invention relating to the food, wherein the food comprises per 100 g: fat 0-5 w %, protein components incl. free amino acids 10-95 w %, carbohydrates incl. sugars 0-10 w %, isosteviol 1-10 w %.

Food, such as another food supplement is considered to be a composition according to an embodiment of the present invention relating to the food, wherein the food comprises per 100 g: protein components incl. free amino acids 10-95 w %, isosteviol 1-10 w %.

Food, such as a fatburner/weight loss capsules/tablets is considered to be a composition (itself or in combination also with no nutritional ingredients like, Chili, Chlorogenic acids etc). Thus, according to an embodiment of the present invention relating to the food, the food comprises per 100 g: other components e.g. protein, herbal extracts, vitamin, minerals 10-95 w %, isosteviol 1-10 w %.

Food, such as a diet shake/soup/ice etc. or a low calorie diet/very low calorie diet/VLCD is considered to be a composition according to an embodiment of the present invention relating to the food, wherein the food comprises per 100 g: fat 1.5-15 w %, protein components incl. free amino acids 10-75 w %, carbohydrates incl. sugars 8-50 w %, isosteviol 0.5-10 w %.

An embodiment of the present invention relates to the food, wherein the drinkable liquid is selected from the group consisting of water, alcohol, oil, milk, juice, and combinations thereof.

An embodiment of the present invention relates to the food, wherein the food is in a form selected from the group consisting of grains, pellets, powder, solution, suspension, dispersion, and slurry.

An embodiment of the present invention relates to the food, wherein the food is distributed in a product selected from the group consisting of a feed, bar, sugar bar, protein bar, powder, gel, beverage, yoghurt, chewing gum, dairy product, sports drink, confectionary product, ice cream, capsule, tablet, sachet, and pouch.

An embodiment of the present invention relates to the food, wherein the food is a poultry feed, a reptile feed, a fish feed, feed for aquacultures, insect feed, worm feed and mammal food/feed, preferably a food for a mammal. Thus, the food can be optimized for different animals both in relation to content, form and size

An embodiment of the present invention relates to the food, further comprising flavours.

In yet an embodiment, the food is selected from the group consisting of gel, beverage, gum, confectionary, droplets, tinctures, homeopathic preparations.

In an embodiment, the food is adapted for sublingual absorption or buccal absorption.

In yet an embodiment, the food provides a daily dosage of Isosteviol (to the subject/animal, such as human or pig) in an amount of at least 0.01 mg isosteviol/kg bodyweight/day, such as at least 0.1 mg isosteviol/kg bodyweight/day, preferably such as at least 0.5 mg isosteviol/kg bodyweight/day, and more preferably such as at least 0.8 mg isosteviol/kg bodyweight/day, or such as around 1 mg isosteviol/kg bodyweight/day, based on the bodyweight of the subject. In the example section, pigs were fed around 1 mg isosteviol/kg bodyweight/day in the groups receiving isosteviol.

In a related embodiment, the food provides a daily dosage of Isosteviol (to the subject/animal, such as human or pig) in an amount of at the most 100 mg isosteviol/kg bodyweight/day, such as at the most 50 mg isosteviol/kg bodyweight/day, such as at the most 20 mg isosteviol/kg bodyweight/day, such at the most 10 mg isosteviol/kg bodyweight/day, preferably such as at the most 5 mg isosteviol/kg bodyweight/day, and more preferably at the most 3 mg isosteviol/kg bodyweight/day, such as around 1 mg isosteviol/kg bodyweight/day, based on the bodyweight of the subject.

Yet a related embodiment the food provides a daily dosage of Isosteviol (to the subject/animal, such as human or pig) in an amount in the range of 0.01 mg-100 mg isosteviol/kg bodyweight/day, such as in the range of 0.1-50 mg isosteviol/kg bodyweight/day, such as in the range of 0.1-20 mg isosteviol/kg bodyweight/day, such as in the range of 0.1-10 mg isosteviol/kg bodyweight/day, preferably such as 0.5-5 mg isosteviol/kg bodyweight/day, and more preferably mg isosteviol/kg bodyweight/day, such as around 1 mg isosteviol/kg bodyweight/day, based on the bodyweight of the subject.

### Use of the Composition or Food Ingredient

A third aspect of the present invention relates to a use of isosteviol, the composition or food ingredient as described herein, or the food as described herein, in/as a food (for an animal).

An embodiment of the present invention relates to the use, wherein the food is fed to an animal.

An embodiment of the present invention relates to the use, wherein the animal is selected from the group consisting of an avian, such as poultry, reptile, fish, insect, worm, and mammal, preferably a mammal.

An embodiment of the present invention relates to the use, wherein the mammal is selected from the group consisting of human, pig, cattle, zebu, donkey, horse, dog, cat, goat, and sheep, preferably a human or a pig.

An embodiment of the present invention relates to the use, wherein the mammal is a racing animal or athlete, subject to exhaustive exercise and the food is distributed to said racing animal or athlete before, during, and/or after the exhaustive exercise.

An embodiment of the present invention relates to the use, wherein the racing animal or athlete is selected from the group consisting of a human athlete, sportsperson, racehorse, and greyhound racing dog.

An embodiment of the present invention relates to the use, wherein the animal is depleted in muscle glycogen.

An embodiment of the present invention relates to the use, wherein the animal is not depleted in muscle glycogen.

As described in here, the present inventors have identified novel uses of isosteviol and compositions comprising isosteviol.

An embodiment of the present invention relates to the use, for increasing the muscle to fat ratio of the animal to which the food is distributed compared to an animal fed with a standard food/feed (free of isosteviol) or a standard food/feed supplemented with protein and/or carbohydrates and/or free amino acids. As shown in example 2, a better uptake is seen when isosteviol is added to the pig feed. Examples 2 and 3 shows that pigs receiving isosteviol and additional protein/amino acids were leaner and more firmly meaty compared to the control group. Also, example 5 shows that the meat/muscle percentage is increased in the groups receiving Isosteviol.

An embodiment of the present invention relates to the use, for decreasing the food/feed conversion ratio (FCR) of the animal to which the food is distributed compared to an animal fed with a standard food/feed or a standard food/feed supplemented with protein and/or carbohydrates and/or free amino acids. Example 3 shows that more feed is taken up by the animals receiving isosteviol, indicating a decreased feed conversion ratio (FCR) (improved uptake).

In this regard, the food/feed conversion ratio (FCR) is defined as the ratio between the amount of food eaten by an animal to the output produced by the animal (e.g. amount of meat produced by a pig, amount of milk produced by a cow etc.). Food that is efficiently taken up and converted by the body of the animal to said output obtains a FCR-value lower than a food having a low uptake in the animal. Thus, lower FCR-values are preferred for most applications. In some sectors, feed efficiency, which is the output divided by the input (i.e. the inverse of FCR), is used.

An embodiment of the present invention relates to the use, for increasing the uptake of proteins and/or carbohydrates and/or free amino acids of the animal compared to an animal fed with a standard food/feed or a standard food/feed supplemented with protein and/or carbohydrates and/or free amino acids. Example 2 shows increased weight gain in pigs receiving isosteviol. An increased weight gain is also seen in pigs receiving feed supplemented with carbohydrates, protein and free amino acids in combination with isosteviol. Notably, pigs receiving feed supplemented with carbohydrates, protein and free amino acids but no isosteviol had a lower weight gain than the control group (without addition of carbohydrates, protein and free amino acids). Without being bound by theory, it is believed that isosteviol allows for better uptake of carbohydrates, protein and free amino acids. When isosteviol is not supplied the pigs does not thrive due to too high amounts of protein and amino acids. Not the least, example 6 shows increased turnover rates of the pigs receiving isosteviol.

An embodiment of the present invention relates to the use, for increasing the growth rate of the animal compared to an animal fed with a standard food/feed or a standard food/feed supplemented with protein and/or carbohydrates and/or free amino acids. Examples 2 and 6 documents increased growth rates.

Other uses according to the present invention are for
Increasing muscle mass in elderly or others where this is relevant such as subjects in physiotherapy treatment after in injuries and the like;
Increasing muscle endurance;
Increasing muscle recovery after exercise;
Muscle recovery after damage such as strains and similar muscular damage;
Reduction in blood sugar in non-diabetic in human and animals.

### Medical Uses

A further aspect of the present invention relates to the composition or food ingredient as described herein, or the food as described herein, for use as a medicament.

Isosteviol affects smooth as well as striated muscles. A composition or food ingredient comprising isosteviol, or food/feed containing isosteviol is considered to be efficient for treating, preventing, and/or alleviation of muscle diseases wherein the muscle mass is to be rebuild, regenerated, and/or replenished. Without being restricted to theory, the rebuilding, regeneration, and/or replenishment of muscle mass by the presence of isosteviol may effectively treat, prevent, and/or alleviate any muscle disease associated with the function of the potassium channel related to the muscles. As shown in example 5, the meat (muscle) percentage is higher in the pigs receiving Isosteviol compared to the controls.

Thus, an embodiment of the present invention relates to the composition or food ingredient as described herein or the food as described herein, for use in the prevention and/or treatment and/or alleviation of a muscle disease/injury, such as fibromyalgia or strains.

A more specific embodiment of the present invention relates to the composition or food ingredient or food for use as described herein, wherein the muscle disease/injury is selected from the group consisting of sclerosis, metabolic syndrome, chronic fatigue syndrome (CFS), fibromyalgia, tendinitis, strains, polymyositis, pre-/post-muscular surgery for faster muscular recovery, hyperkalemic periodic paralysis, and hyperkalemic disorders where symptomatic treatment is necessary.

Isosteviol also affects the intestinal system e.g. by influencing the smooth muscle layer of the intestine.

Thus, an embodiment of the present invention relates to the composition or food ingredient as described herein or the food as described herein, for use in the prevention and/or treatment and/or alleviation of a digestive disorder.

An embodiment of the present invention relates to the composition or food ingredient or food for use as described herein, wherein the digestive disorder is selected from the group consisting of a Chrohn's disease such as morbus Crohn, diarrhea, ulcerative colitis, irritable bowel syndrome, and irritable bowel disease. In a preferred embodiment, the digestive disorder is diarrhea, such as diarrhea caused by high intake of protein, free amino acids and/or carbohydrates (see example 4).

An embodiment of the present invention relates to the composition or food ingredient as described herein or the food as described herein, for use as a blood sugar lowering agent.

An embodiment of the present invention relates to the composition or food ingredient as described herein or the food as described herein, wherein isosteviol is administered to a subject (animal(s), such as human or pig) in need thereof in an amount of at least 0.01 mg isosteviol/kg bodyweight/day, such as at least 0.1 mg isosteviol/kg bodyweight/day, preferably such as at least 0.5 mg isosteviol/kg bodyweight/day, and more preferably such as at least 0.8 mg isosteviol/kg bodyweight/day, or such as around 1 mg isosteviol/kg bodyweight/day, based on the bodyweight of the subject. In the example section, pigs were fed around 1 mg isosteviol/kg bodyweight/day in the groups receiving isosteviol.

In a related embodiment, isosteviol is administered to a subject (animal, such as human or pig) in need thereof in an amount of at the most 100 mg isosteviol/kg bodyweight/day, such as at the most 50 mg isosteviol/kg bodyweight/day, such as at the most 20 mg isosteviol/kg bodyweight/day, such at the most 10 mg isosteviol/kg bodyweight/day, preferably such as at the most 5 mg isosteviol/kg bodyweight/day, and more preferably at the most 3 mg isosteviol/kg bodyweight/day, such as around 1 mg isosteviol/kg bodyweight/day, based on the bodyweight of the subject.

Yet a related embodiment of the present invention relates to the composition or food ingredient as described herein or the food as described herein, wherein isosteviol is administered to a subject in need thereof in an amount in the range of 0.01 mg-100 mg isosteviol/kg bodyweight/day, such as in the range of 0.1-50 mg isosteviol/kg bodyweight/day, such as in the range of 0.1-20 mg isosteviol/kg bodyweight/day, such as in the range of 0.1-10 mg isosteviol/kg bodyweight/day, preferably such as 0.5-6 mg isosteviol/kg bodyweight/day, and more preferably 0.5-3 mg isosteviol/kg bodyweight/day, such as around 1 mg isosteviol/kg bodyweight/day, based on the bodyweight of the subject.

In yet an embodiment, isosteviol is administered to a subject in need thereof in an amount in the range of 0.1-10 mg isosteviol/kg bodyweight/day, preferably 0.5-6 mg isosteviol/kg bodyweight/day, and more preferably 0.5-3 mg isosteviol/kg bodyweight/day. It is of course to be understood that amounts disclosed for one aspect of the invention may also be applicable to another aspect of the invention.

Further embodiments relate to the compositions, food ingredients and food for
Treatment and/or alleviation and or prevention of muscle damage and/or atrophia;
Treatment and/or alleviation and or muscle recovery from injuries.
Treatment and/or alleviation and or prevention of fatty liver disorders.

### Process for Producing a Food

An even further aspect of the present invention relates to a process for manufacturing of a food as described herein, the process comprising the steps of:
I. providing the composition or food ingredient as described herein,
II. bringing the composition or food ingredient into contact with one or more additional food component, such as a standard food and/or a supplementary food,
III. optionally, adding the ingredient to a capsule, tablet, sachet, or pouch, gel, beverage, gum, confectionary, droplets, tinctures, homeopathic preparations, or preparations adapted for sublingual absorption or buccal absorption.

An embodiment of the present invention relates to the process, wherein the additional food component is selected from the group consisting of standard food, a supplementary food or combinations thereof.

An embodiment of the present invention relates to the process, wherein the standard food comprises material from one or more of crops or parts thereof.

An embodiment of the present invention relates to the process, wherein the supplementary food comprises any one or more material selected from the group consisting of crude fibre, crude ash, crude protein, crude fat, nutrient, inorganics, enzyme, free amino acid, food additive, soya bean, and sunflower seed.

An embodiment of the present invention relates to the process, wherein the composition or food ingredient is brought into contact with one or more additional food component by a method selected from the group consisting of mixing, admixing, agitating, blending, and stirring.

### Feeding System

An additional aspect of the present invention relates to a feeding system comprising:
I. the composition or food ingredient as described herein,
II. a supplementary food and/or a standard food; and
III. optionally, instructions for manufacturing a food as described herein by the process as described herein.

An embodiment of the present invention relates to the feeding system, wherein the composition or food ingredient, the supplementary food, the standard food is provided in bags, containers, receptacles, jars, and/or sacks.

### Other Aspects of the Invention

In a further aspect of the invention relates to a method for
Increasing muscle to fat ratio in an animal; and/or
Decreasing the food/feed conversion ratio (FCR) in an animal; and/or
Increasing the uptake of proteins and/or carbohydrates and/or free amino acids in an animal; and/or
Increasing the growth rate of an animal;
   the method comprising administering a food according to the invention to an animal in need thereof. Preferably, the animal is a human or a pig.

Yet a further aspect of the invention relates to a method for
prevention and/or treatment and/or alleviation of diarrhea in an animal; and/or
prevention and/or treatment and/or alleviation of muscle diseases/injuries in an animal; and/or
prevention and/or treatment and/or alleviation of digestive disorders in an animal;
   the method comprising administering a food according to the invention to an animal in need thereof. Preferably, the animal is a human or a pig.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1—Materials and Methods—Isosteviol, Animals and Feeds

### Isosteviol:

Isosteviol in 97% purity was produced in-house. Isosteviol may be produced by acid-catalysed extraction of Stevia rebaudiana L. Preferably at a pH below 5.5.

### Pigs:

The pigs were of the type Danish Landrace Age approx. 90 days, gender equally distributed. They weighted approximately 30 kg when delivered and had been fed with a normal conventional diet for piglets until delivery.

### Feeds:

Standard feed is home-blended by the farmer with the ratio: Wheat 55 w %, Barley 35 w %, Rye 10 w %.
   Nutritional profile/100 g:
      Nutritional content: 1313 KJ
      Fat: 1.8 g
      Carbohydrate: 68.9 g
      Of which sugar: 1.4 g
      Fibres: 15.8 g
      Protein: 12.8 g
Supplementary feed: The Supplementary feed was distributed as a “Semi Soja 1920” composition (from DLG), which is a supplementary feed high in protein, carbohydrate, and free amino acids. The composition of the supplementary feed is shown in Table 1.

TABLE 1
Supplementary feed

| | Value | Unit |
|---|---|---|
| Components: | | |
| Soybean meal | 55.30 | w % |
| Sunflower seed | 18.50 | w % |
| Calcium carbonate | 6.34 | w % |
| Oat | 5.00 | w % |
| Barley | 4.80 | w % |
| Rock salt | 2.40 | w % |
| L-lysine sulphate | 2.34 | w % |
| Monocalcium phosphate | 1.94 | w % |
| Palm fat | 1.30 | w % |
| Vitamin mix. premix | 0.98 | w % |
| Threonine | 0.60 | w % |
| DL-Methionine | 0.20 | w % |
| Xylanase enzyme | 0.19 | w % |
| Phytase enzyme | 0.11 | w % |
| Vitamin mix. premix: | | |
| Fe, Iron(II) sulphate monohydrate | 412 | mg |
| Cu, Cobber(II) sulphate, pentahydrate | 49 | mg |
| Mn, Manganese(II) oxide | 206 | mg |
| Zn, Zinc oxide | 270 | mg |
| I, Calcium iodate | 1.08 | mg |
| Se, sodium selenite | 1.57 | mg |
| A-vitamin | 20580 | i.u. |
| D3-vitamin | 2058 | i.u. |
| E-vitamin, all-rac-alpha-tocopheryl acetate | 210 | i.u. |
| L-lysine sulphate and by-products | 13.0 | g |
| DL-methionine, technically pure | 2.0 | g |
| L-threonine | 5.9 | g |
| Propyl gallate | 19.1 | mg |
| 6-phytase | 5500 | units |
| Endo-1,4-beta-xylanase | 15200 | units |
| Analytical constituents: | | |
| Crude protein | 34.4 | w % |
| Crude oils and fats | 3.6 | w % |
| Crude fibre | 6.9 | w % |
| Crude ashes | 15.1 | w % |
| Lysine | 31.0 | g/kg |
| Methionine | 7.0 | g/kg |
| Calcium | 30.7 | g/kg |
| Phosphor | 10.4 | g/kg |
| Sodium | 9.6 | g/kg |
| Zinc | 318 | mg/kg |

i.u. International unit for vitamins.

### Example 2—Food Comprising Isosteviol Increases the Growth Rate of Pigs

### Aim of Study

To establish if a feed comprising isosteviol increases the growth rate of pigs and/or impact on the muscle (meat)/fat ratio of the pig.

### Materials and Methods:

TABLE 2
Groups and food provided

| Test groups | Number of pigs | Feed compositions |
|---|---|---|
| Control (CG) | 5 pigs | Standard feed incl. standard dose of |
| | | supplementary feed 204 g/kg |
| | | grainmix |
| Protein (CPG) | 5 pigs | Standard feed + additional 10 w % |
| | | supplementary feed (compared to CG |
| | | group) |
| Isosteviol and | 9 pigs | Standard feed + additional 10 w % |
| Protein (IPG) | | supplementary feed (compared to CG |
| | | group) + isosteviol in 1 mg/kg/day |
| | | based on the bodyweight of the pig |
| Isosteviol only | 8 pigs | Standard feed incl. standard dose of |
| (IG) | | supplementary feed. + isosteviol in 1 |
| | | mg/kg/day based on the bodyweight |
| | | of the pig |

The pigs were randomly distributed in the five pigsties when trial started. Food and water were provided ad libitum (see Table 2). The farmer was not aware of which groups received which additional feed, since all feeds were masked to look the same and the CG group received a bag similar to the other groups.

Every other week the pigs were weighed, and their growth were noted. The pigs were attended too equally.

Timeframe: Trial started after delivery of the 30 kg pigs and was planned to end when the pigs weighed around 115-118 kg, the weight at trial-end was however around 120 kg (11 weeks). Total time for production is 72 days.

The food intake by each pig during the trial is described in Table 3.

TABLE 3
Food intake by each pig during the trial

| | Feed | | |
|---|---|---|---|
| | Total ^{a)} | sup. feed ^{b)} | Isosteviol in 1 pig ^{c)} |
| Weeks | (kg/pig/day) | (g/pig/day) | (mg/pig/day) |
| 1 | 1.300 | 27.30 | 30.00 |
| 2 | 1.465 | 30.77 | 37.35 |
| 3 | 1.630 | 34.23 | 44.70 |
| 4 | 1.795 | 37.70 | 52.05 |
| 5 | 1.960 | 41.16 | 59.40 |
| 6 | 2.125 | 44.63 | 66.75 |
| 7 | 2.290 | 48.09 | 74.10 |
| 8 | 2.455 | 51.56 | 81.45 |
| 9 | 2.620 | 55.02 | 88.80 |
| 10 | 2.785 | 58.49 | 96.15 |
| 11 | 2.950 | 61.95 | 103.50 |

Estimated average amount of feed per day consumed by one pig,
^{a)} of group CG, CPG, IPG or IG,
^{b)} of group CPG or IPG,
^{c)} of group IPG or IG.

### Results

TABLE 4
Results obtained during and after trial

| | Average weight (kg) | | | Net growth vs. | Net growth vs. |
|---|---|---|---|---|---|
| Pigs | start | week 8 | Increase | CG group | CPG group |
| CG | 34.80 | 98.80 | 64.0 | 100.0 | 101.88 |
| CPG | 32.40 | 95.20 | 62.80 | 98.1 | 100.00 |
| IPG | 33.11 | 99.78 | 66.67 | 104.0 | 105.80 |
| IG | 35.00 | 99.50 | 64.50 | 100.8 | 102.71 |

Table 4 summarizes the results obtained during and after the trial period.

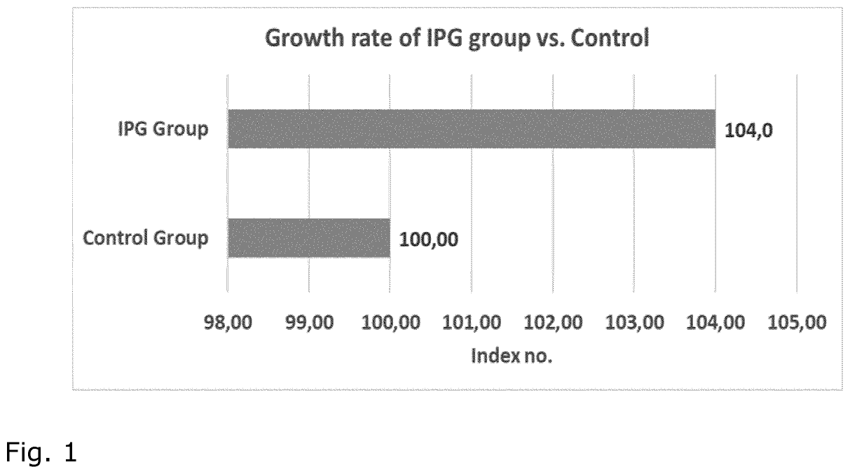

Growth comparison between CG group and IPG group is shown in FIG. 1. It can be seen that the IPG group had a clear increased weight gain compared to the control group (CG).

Growth comparison between CPG group and IPG group is shown in FIG. 2. It can be seen that the IPG group had a clear increased weight gain compared to the CPG group. Interestingly the difference here is higher than for the comparison shown in FIG. 1. Without being bound by theory, it is believed that the pigs in the CPG group are not able to metabolize the increased amount of protein in the absence of isosteviol. It is well-known that by simply increasing protein content of a feed does not result in weight gain, and can in fact result in e.g. diarrhea. It is noted that this effect is known also from humans.

Growth comparison between control group (CG) and isosteviol group (IG) is shown in FIG. 3. It can be seen that the IG group had a clear increased weight gain compared to the Control (CG) group. Thus, also without the addition of a supplementary feed (containing additional protein, carbohydrates and free amino acids) the addition of isosteviol results in an increased weight gain.

### Conclusion

Addition of isosteviol to the feed, results in an increased weight gain of the pigs. The effect is more pronounced if the feed is supplemented with proteins/free amino acids and carbohydrate. It should be recalled that by just adding increased levels of protein/free amino acids and carbohydrate to a feed in the absence of isosteviol may result in diarrhea (se example 4).

In sum, the results show a surprisingly high increase in growth of the IPG group, which received Isosteviol plus protein compared with the CG group. Thus, it was found that the IPG group can utilize the combined intake of extra protein and Isosteviol efficiently in their body to increase growth.

Results also demonstrate, that the use of extra quantities of protein mixes (supplementary feed) that farmers often use to increase growth and meat percentage in the pigs resulted in lower growth. This corresponds with the general knowledge that too much protein and/or amino acids can cause both diarrhea or bad well-being for the animals and thus poor growth or worse health conditions.

Surprisingly, when adding more supplementation together with Isosteviol it was found that the pigs thrived better and could utilize the extra nutritional supplementation specifically protein, amino acids and carbohydrates to increase meat production in the individual pigs (increased muscle/fat ratio).

A better growth in the IG group compared to the CG was also identified. Being a smaller percentage though, which is believed consistent with lacking the extra nutrients to use the Isosteviol to build more muscles. Pigs feed today are very closely calculated to the optimum in correlation of growth and meat production, however, without being bound by theory it is believed that the data shows that it is possible to gain more meat from one pig using Isosteviol to increase the muscle build pace.

Isosteviol alone and/or in combination with protein, free amino acids and carbohydrate provides a higher muscle/fat ratio percentage in the animal and humans thus providing a lower bodyfat content. These results are seen even with normal exercise within the pigsty.

Isosteviol alone and/or in combination with protein, free amino acids and carbohydrate provides a body profile aiming more towards more muscles and a leaner body with a lower bodyfat content.

Isosteviol alone and/or in combination with protein, free amino acids and carbohydrate gives less stool and thus compared with the growth this means that we get the exceeding growth not only through the extra nutrition, but also because of a better food/feed conversion ratio.

All data also suggest that Isosteviol alone and/or in combination with protein, free amino acids and carbohydrate can be used in different appliances of the feed and food area of use. Data also suggesting Isosteviol alone and/or in combination with protein, free amino acids and carbohydrate within the treatment of different muscle diseases as well as digestive areas.

Notably, the pigs were not subjected to extensive/unusual exercise during the trial. Thus, the pigs were not glycogen depleted.

### Example 3—Environmental Advantages of Feed Comprising Isosteviol Aim of Study

To establish if there are environmental advantages of feeds comprising isosteviol. An environmental advantage is e.g. increased overall uptake of the provided feed, thus less of the feed ends up as manure/stool, which the farmer has to get rid of e.g. by distributing it on fields.

### Materials and Methods

See example 1.

Overall uptake of feed was determined by the amount of generated manure by the different groups.

As shown in FIG. 4, it can be seen from the top pictures that much more manure has been generated from the pigs, e.g. visualized by how messy the pigs look.

Furthermore, the farmer has repeatedly noted the following:

Pigsty for the CPG group excretes extensive stool (as seen in FIG. 4) compared to the groups receiving additional protein and carbohydrates together with Isosteviol, the IPG group who received the same quantities of protein, amino acids and carbohydrates.

The farmer, furthermore noted that the pigs in IPG group were leaner and more firmly meaty compared to the control group. This indicates a higher muscle to fat ratio compared to the controls.

### Conclusion

The addition of isosteviol to the feed results in a higher overall uptake of the feed, thus, providing a clear environmental advantage and not the least economical advantage.

Further, the more meaty appearance of the pigs receiving isosteviol indicates a higher muscle to fat ratio compared to the controls.

Also, stool analysis indicate that the stool from the pigs receiving Isosteviol were more firm and also contained less total nutrients. Preliminary stool analysis indicate that Isosteviol decreases the amount of nutritional ingredients in stool by approx. 6% considering the quantity of manure discharged (data not shown).

### Example 4—Feed Comprising Isosteviol Prevents and/or Treats Pigs From Diarrhea

Aim of study

To determine if feed comprising isosteviol prevents and/or treats pigs from diarrhea.

### Material and Methods

See example 1.

### Results

The group receiving isosteviol (IPG and IG) had a much firmer stool compared to the control groups (CG and CPG). This difference was more pronounced in the control group receiving addition protein and amino acids (CPG) compared to the group which also received isosteviol (IPG).

The data was confirmed by the farmer attending the pigs.

That a firm stool is present in the groups receiving isosteviol can actually also be seen in FIG. 4, where the pigs in the control group are much more messier indicating increased amount of stool but also a much looser stool, indicative of diarrhea.

### Conclusion

Feed supplemented with isosteviol produced a much firmer stool. These data clearly indicates that isosteviol may prevent or treat diarrhea, especially in animals receiving a diet high in protein and/or free amino acids and/or carbohydrates.

Further Isosteviol alone and/or in combination with protein, free amino acids and carbohydrate provides a protection against diarrhea. This will be of great importance also looking into small piglets production when going through the digestive change from the breastmilk to starting processing feed as zinc replacement. By improving the digestive system the immune system will be enhanced. The gastrointestinal system is overall important for a strong immune system hosting approx. 70% of the entire immune system. This is also seen demonstrated as the stool quality in the group fed with Isosteviol is considerably better.

All data also suggest that Isosteviol alone and/or in combination with protein, free amino acids and carbohydrate can be used in different appliances of the feed and food area of use. Data also suggesting Isosteviol alone and/or in combination with protein, free amino acids and carbohydrate within the treatment of different muscle diseases as well as digestive areas.

### Example 5—Meat Percentage of Tested Groups

Aim of study

To determine if the meat (muscle) percentage of the animals were changed in the tested groups.

### Materials and Methods

See also previous examples.

Meat percentage is measured when the pig is dead. The body is prepared and send to an automatic equipment that measures fat/meat thicknesses based on ultrasound measurements called an Autoform. The equipment is certified by third party and the meat percentages in this study was performed by Tican A/S at the slaughterhouse in Brdrup.

Increased meat (muscle) percentage can also be considered to correspond to an increased muscle/fat ratio.

### Results

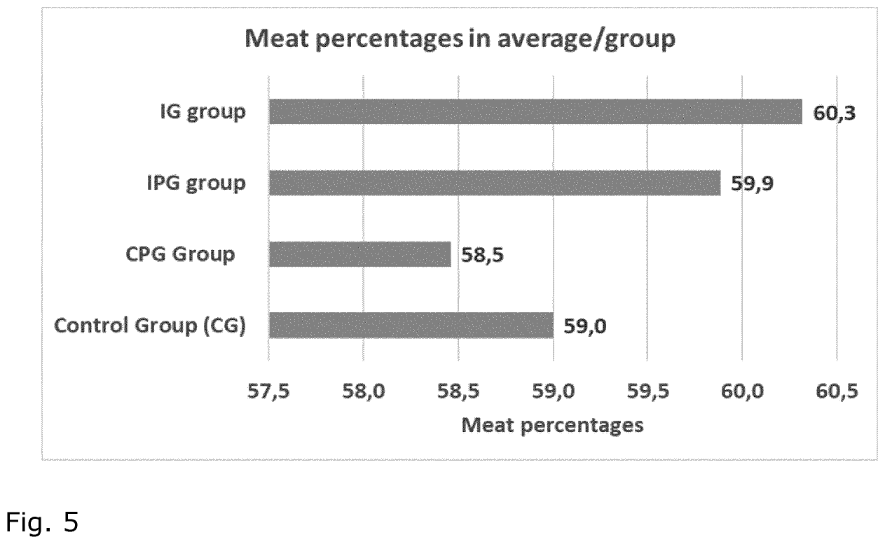

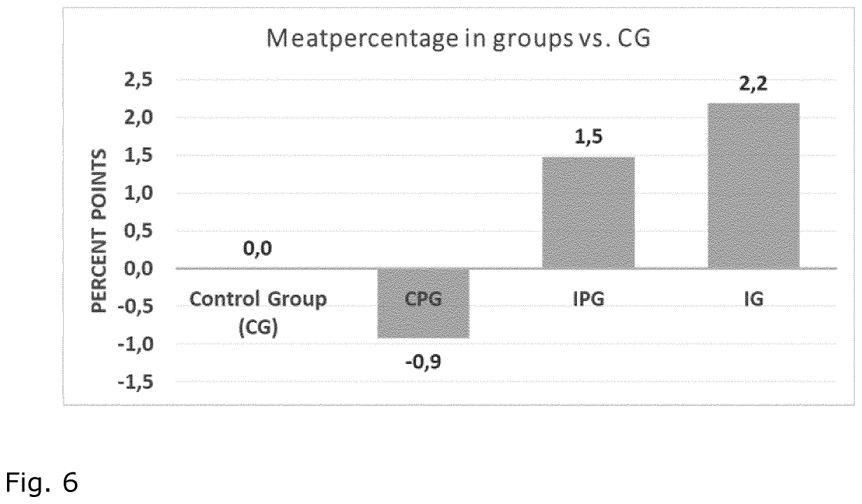

As described above, it was found that the IG group did only have a slight but consistent increase in average bodyweight (see table 4 above and FIG. 3), however a very significant increase of the muscle weight also defined as an increase in the muscle/fat ratio in the body was identified (FIG. 5). Thus, it was surprisingly found that it was possible to improve the muscle/fat ratio by adding Isosteviol alone to the diet, without further restrictions in diet or fitness habits et cetera. Also important, it can be seen that by simply adding supplementary feed (without adding Isosteviol) to the standard feed shows no effect on the meat percentage (or and muscle/fat ratio) (FIG. 6).

### Conclusion

From these findings, it can be concluded that adding isosteviol to a food product can increase the amount of muscles and therefore also increase muscle/fat ratio.

Without being bound by theory, by adding also exercise, will add positively to the conversion from fat to muscles/meat at a higher rate.

It was also found that the IPG group despite having increased their average bodyweight in the group also increased their muscle/fat ratio to a significant degree. It can thus also be concluded that by adding Isosteviol and a mix of carbohydrate, proteins and amino acids, it is possible to gain more muscles.

Surprisingly and interestingly using Isosteviol alone or in combination with carbohydrate, protein and amino acids (in here in the form of a supplementary feed) may thus work as a weight-losing or muscle-gaining ingredient in both animals and humans.

Surprisingly the results demonstrate that people/animals to some degree exercising, like open air grown animals will gain even more effect from Isosteviol alone or in combination with carbohydrate, protein and/or amino acids, since they use their muscles considerably more and thus benefit even more from the findings in muscle/fat conversion ratio.

### Example 6—Turnover Rates

### Aim of Study

To determine if the turnover rates of the pigs are increased when Isosteviol is added to the feed.

### Materials and Methods

See also previous examples.

Turnover rates of the pigs was defined by the time (days) it takes to grow the pigs from approx. 30 kg to around 118 kg. Thus, turnover time is the time it takes to grow the pigs from approx. 30kg to their optimum slaughter weight. The faster the turnover rate is, the faster the pigs goes through the process and consequently this is the determination factor on how many pigs the farmer can deliver to the slaughterhouse within a year.

Thus, the turnover rate is as in every company the essential factor for effective the production is and thus directly impacting the operating margin.

### Results

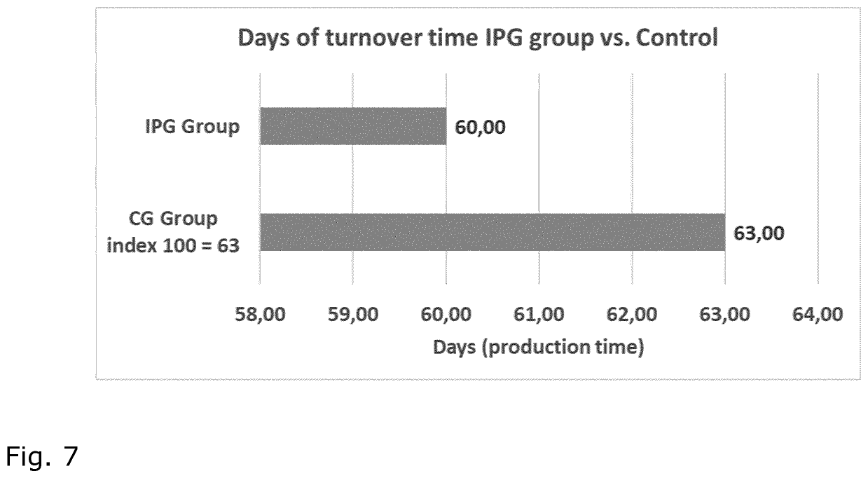

When comparing the CG group to the IPG group, the turnover rate is increased by approx. 4.8% in the IPG group (FIG. 7) by decreasing the production time.

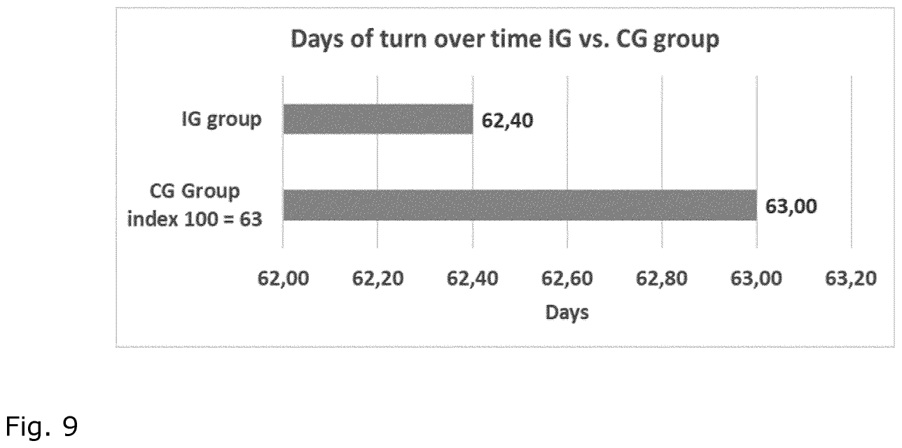

When comparing the CG group to the IG group, the turnover rate increased by approx. 1% in the IG group (FIG. 9).

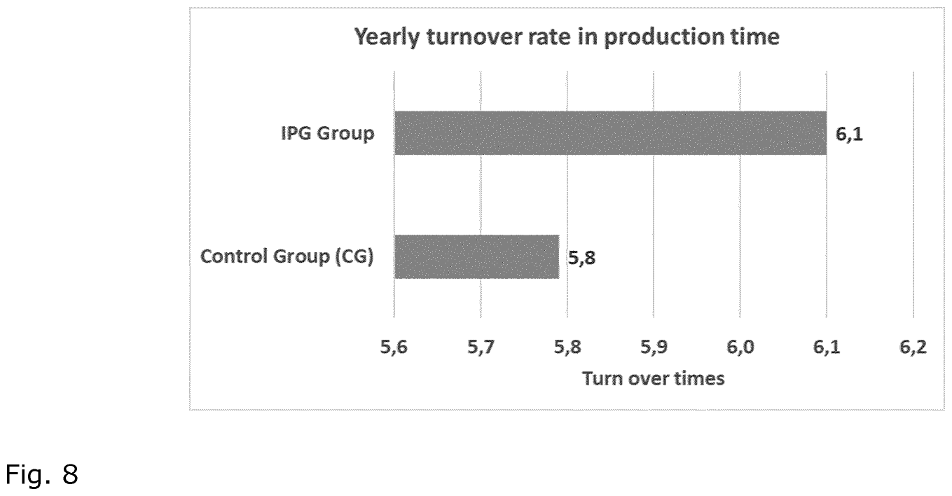

In FIG. 8 it is shown that the yearly turnover in production will be significantly increased in the IPG group compared to the CG group. This corresponds to an increased turnover rate for the IPG group compared to CG group by around 4.8%.

When comparing the CG group to the IG group, the yearly turnover in production was also increased by 0.1% (data not shown).

### Conclusion

Lowering the turnover rate of pigs brings a significantly increased value to the farmer and furthermore adds to a more positive environmental profile for the farmers by reducing the environmental pressure (per pig), since period the pigs do eat, drink water and excrete any stool is considerably reduced.

In sum, the increased turnover time (and increased yearly turnover in production) leads to the conclusion that feed comprising Isosteviol e.g. supplemented with carbohydrate and/or protein and/or amino acids will increase food/feed utilization.

Stool sample analysis also supports this finding (data not shown, see also example 3).

### Example 7—Food Composition According to the Invention

Below are provided a range of food/feed which should be considered non-limiting examples of food/feed according to the present invention.

### Composition 1—Pigs/Sows:

| | % (min-max) per |
|---|---|
| Feed | kg Dry-matter |
| Fat | 1-8% |
| Protein not only as crude protein from example | 3.00-30% |
| sunflower, soy etc., free amino acids | |
| carbohydrates not only: Grain mix, corn, sugar beet | 50-95% |
| molasses | |
| isosteviol | <5% |
| Amino acids not only but also: Lysine, Methionine, | 0.3-10% |
| tryptophan, Leucine, Histidine, Valine | |

### Composition 2—Grower/Slaughter Pigs:

| | % (min-max) per |
|---|---|
| Feed | kg Dry-matter |
| Fat | 0.5-10% |
| Protein not only as crude protein from example | 1-20% |
| sunflower, soy etc., free amino acids | |
| carbohydrates not only: Grain mix, corn, sugar beet | 15-40% |
| molasses | |
| isosteviol | <5% |
| Amino acids not only but also: Lysine, Methionine, | 0.3-10% |
| tryptophan, Leucine, Histidine, Valine | |

### Composition 3—Piglets

| | % (min-max) per |
|---|---|
| Feed | kg Dry-matter |
| Fat | 1-8% |
| Protein not only as crude protein from example | 3-33% |
| sunflower, soy etc., free amino acids | |
| carbohydrates not only: Grain mix, corn, sugar beet | 50-95% |
| molasses | |
| Isosteviol | <5% |
| Amino acids not only but also: Lysine, Methionine, | 0.3-10% |
| tryptophan, Leucine, Histidine, Valine | |

### Composition 4—Food and/or Supplementary Feed for Grower Pigs/Piglets

| | % (min-max) per |
|---|---|
| Feed/Feed supplement | kg Dry-matter |
| Fat | 2-12% |
| Protein not only as crude protein from example | 22-65% |
| sunflower, soy etc., free amino acids | |
| carbohydrates not only: Grain mix, corn, sugar beet | 3-20% |
| molasses | |
| Isosteviol | <5% |
| Free Amino acids not only but also: Lysine, | 25-65% |
| Methionine, tryptophan, Leucine, Histidine, Valine | |

### Composition 5—Fish

| | % (min-max) per |
|---|---|
| Feed | kg Dry-matter |
| Fat | 10-55% |
| Protein not only as crude protein from example | 25-65% |
| sunflower, soja etc., free amino acids | |
| carbohydrates not only: Grain mix, corn, sugar beet | 5-35 |
| molasses | |
| isosteviol | <5% |

### Composition 6—Racehorse and Other High Performing Animals

| | % (min-max) per |
|---|---|
| Feed | kg Dry-matter |
| Fat | 2.00-10% |
| Protein not only as crude protein from example | 3-22% |
| sunflower, soy etc., free amino acids | |
| carbohydrates not only: Grain mix, corn, sugar beet | 15-95% |
| molasses | |
| isosteviol | <5% |

### Composition 7—Working Animals

| | % (min-max) per |
|---|---|
| Feed | kg Dry-matter |
| Fat | 2.00-25% |
| Protein not only as crude protein from example | 6-42% |
| sunflower, soy etc., free amino acids | |
| carbohydrates not only: Grain mix, corn, sugar beet | 15-84% |
| molasses | |
| isosteviol | <8% |

### Composition 8—Vitamin Water/Sports Water

| Food | % (min-max) per 100 g |
|---|---|
| Fat | 0-2% |
| Protein components incl. free amino acids | 0-10% |
| carbohydrates incl. sugars | 1-30% |
| isosteviol | <5% |

### Composition 9—Sports Gel

| Food | % (min-max) per 100 g |
|---|---|
| Fat | 0-10% |
| Protein components incl. free amino acids | 0-10% |
| carbohydrates incl. sugars | 10-95% |
| isosteviol | 1-8% |

### Composition 10—Protein Shake

| Food | % (min-max) per 100 g |
|---|---|
| Fat | 0-10% |
| Protein components incl. free amino acids | 0-10% |
| carbohydrates incl. sugars | 5-80% |
| isosteviol | 1-8% |

### Composition 11—Supplement Shake for Elderly or Malnutritioned/Undernutritioned People

| Food | % per 100 ml |
|---|---|
| Fat | 0-20% |
| Protein components incl. free amino acids | 5-30% |
| carbohydrates incl. sugars | 5-80% |
| isosteviol | 1-8% |

### Composition 12—Supplement Fibershake

| Food | % per 100 ml |
|---|---|
| Fat | 0-20% |
| Protein components incl. free amino acids | 3-30% |
| carbohydrates incl. sugars | 5-80% |
| fibers | 1-15% |
| isosteviol | 1-8% |

### Composition 13—Protein Powder

| Food | % per 100 g |
|---|---|
| Fat | 0-5% |
| Protein components incl. free amino acids | 10-95% |
| carbohydrates incl. sugars | 0-20% |
| isosteviol | 1-10% |

### Composition 14—Food Supplement

| Food | % per 100 g |
|---|---|
| Fat | 0-5% |
| Protein components incl. free amino acids | 10-95% |
| carbohydrates incl. sugars | 0-10% |
| isosteviol | 1-10% |

### Composition 15—Food Supplement

| Food | % per 100 g |
|---|---|
| Protein components incl. free amino acids | 10-95% |
| isosteviol | 1-10% |

### Composition 16—Fatburner/Weight Loss Capsules/Tablets

| Food | % per 100 g |
|---|---|
| other components may be protein, herbal extracts, | 10-95% |
| vitamin, minerals | |
| isosteviol | 1-10% |

### Composition 17—Diet Shakes/Soups/Ice etc. Low Calorie Diet/Very Low Calorie Diet/VLCD

| Food | % per 100 g |
|---|---|
| Fat | 1.5-15% |
| Protein components incl. free amino acids | 10-75% |
| carbohydrates incl. sugars | 8-50% |
| isosteviol | 0.5-10% |

### Composition 18—Food Ingredient—Isosteviol Powder

| Ingredient | % per 100 g |
|---|---|
| isosteviol | 80-99.9% |

For all the compositions, it is of course to be understood that the amounts always have to be adjusted according to a daily recommend intake. For example, the skilled person would know how to adjust compositions and intake to a recommended daily intake, such as daily recommended intake of isosteviol.

### Example 8—Larger Pig Trial, Weight Gain and Feed Conversion Ratio (FCR) Study

Trial with 360 barrows and female piglets (DanBred×Duroc; initial BW of about 6.8 kg).

### Materials and Methods

Age when trial started approx. 7 days and trial ended after 42 days. They weighed approx. 7 kg initially and have been feed as per normal conventional diet for piglets until delivery.

### Trial Site:

Livestock feed tests Denmark, trial stable: Pebersmarkvej 31, 6372 Bylderup-Bov, Denmark.

Basal diets as per standard industry feed:
Prestarter diet for post-weaning piglets: 7.0 to 9.0 kg BW
Starter diet for piglets: 9.0 to 15.0 kg BW
Grower diet for piglets: 15.0 to 30.0-35.0 kg BW.

Isosteviol >97% was produced in-house. Isosteviol may be produced by acid-catalysed extraction of Stevia rebaudiana L. Preferably at a pH below 5.5.

### Groups:

Negative control: No isosteviol
Treatment 1 (IS10): 1% isosteviol mg/kg Bodyweight (BW)
Treatment 2 (IS13): 1.3% isosteviol mg/kg Bodyweight (BW)

### Feeds:

The pigs were fed standard diets supplemented with Isosteviol as outlined above.

IsoSteviol Premix 0.71% was produced in Vilofoss Premix factory, and included at 0.29/0.37, 0.25/0.33% (by weight) in complete feed to reach 1/1.3 mg IsoSteviol/kg BW (bodyweight).

The piglets were randomly distributed in 10 pigsties per treatment when trial started. Food and water were provided ad libitum. The farmer was not aware of which groups received which additional feed, since all feeds were masked to look the same and the control group received a bag similar to the other groups.

### Results

### Weight Gain:

Body weight [kg]
Absolute Values; Data as Available
STATISTICS (Conf. Level = 95.0%)

| | SD 0 | SD 10 | SD 21 | SD 42 |
|---|---|---|---|---|
| NegControl | | | | |
| Mean | 7.866 | 11.570 | 15.413 | 30.088 |
| Std Dev | 1.1420 | 1.7653 | 3.0864 | 6.6112 |
| Min | 5.000 | 6.300 | 7.800 | 15.400 |
| Lo Quar | 7.150 | 10.600 | 13.000 | 26.300 |
| Median | 7.875 | 11.500 | 15.600 | 30.700 |
| Up Quar | 8.900 | 12.900 | 17.300 | 34.300 |
| Max | 9.850 | 16.500 | 23.900 | 43.600 |
| Valid N | 90 | 89 | 89 | 89 |
| Treatment1 | | | | |
| Mean | 7.868 | 11.568 | 15.783 | 32.660 |
| Std Dev | 1.1313 | 1.7636 | 3.2915 | 6.2647 |
| Min | 5.300 | 6.400 | 3.400 | 15.100 |
| Lo Quar | 7.100 | 10.400 | 13.600 | 28.700 |
| Median | 7.850 | 11.550 | 15.900 | 33.200 |
| Up Quar | 9.000 | 12.900 | 18.200 | 36.700 |
| Max | 9.900 | 15.600 | 23.300 | 44.300 |
| Valid N | 90 | 90 | 90 | 90 |
| Treatment2 | | | | |
| Mean | 7.865 | 11.574 | 15.558 | 32.293 |
| Std Dev | 1.1388 | 1.8017 | 2.7793 | 5.7830 |
| Min | 5.250 | 6.100 | 8.600 | 17.800 |
| Lo Quar | 7.100 | 10.400 | 13.600 | 29.000 |
| Median | 7.875 | 11.500 | 15.600 | 32.000 |
| Up Quar | 9.000 | 12.800 | 17.500 | 36.100 |
| Max | 9.850 | 16.200 | 21.200 | 47.100 |
| Valid N | 90 | 90 | 89 | 89 |

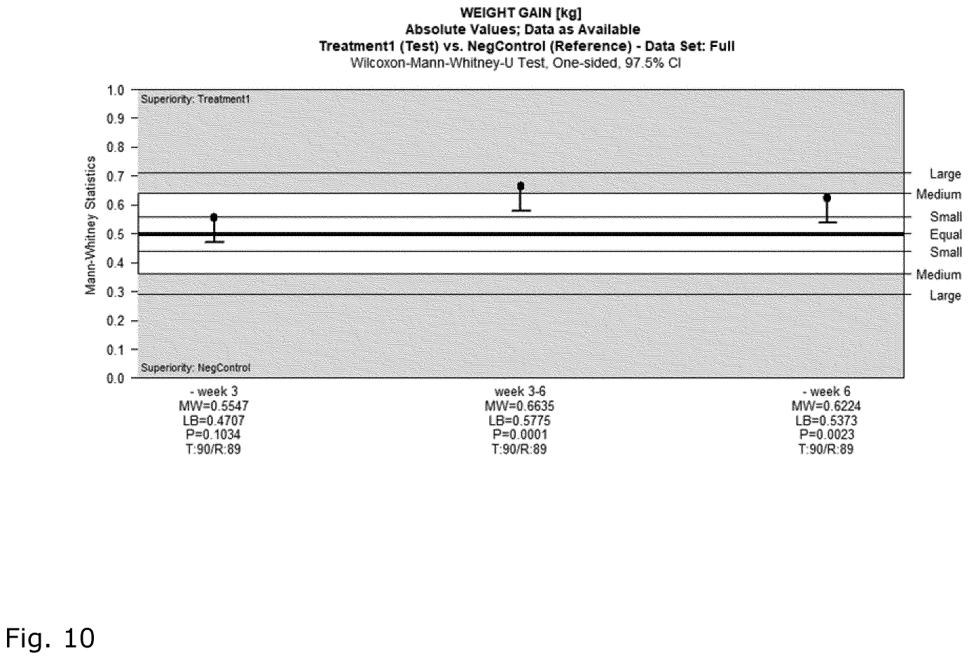

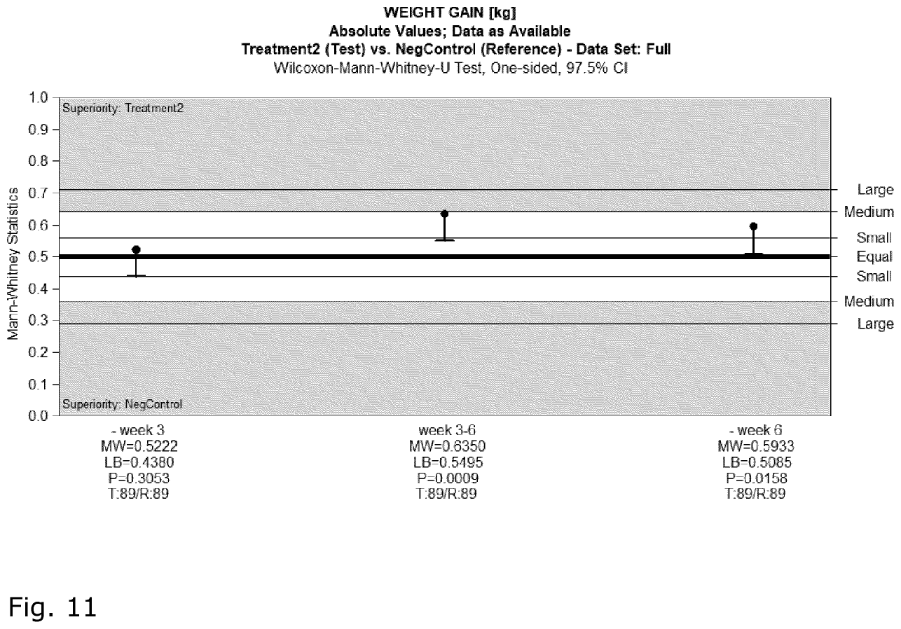

As also shown in FIGS. 10 and 11, the pigs receiving treatment 1 and treatment 2 (both including Isosteviol) had a significant higher weight gain after the 42 days trial.

### Feed Conversion Ratio (FCR):

Absolute Values; Data as Available
STATISTICS (Conf. Level = 95.0%)

| | SD 42 |
|---|---|
| NegControl | |
| Mean | 1.525 |
| Std Dev | 0.1671 |
| Min | 1.330 |
| Lo Quar | 1.370 |
| Median | 1.510 |
| Up Quar | 1.710 |
| Max | 1.760 |
| Valid N | 10 |
| Treatment1 | |
| Mean | 1.399 |
| Std Dev | 0.0671 |
| Min | 1.320 |
| Lo Quar | 1.340 |
| Median | 1.380 |
| Up Quar | 1.420 |
| Max | 1.520 |
| Valid N | 10 |
| Treatment2 | |
| Mean | 1.393 |
| Std Dev | 0.0646 |
| Min | 1.340 |
| Lo Quar | 1.340 |
| Median | 1.360 |
| Up Quar | 1.440 |
| Max | 1.500 |
| Valid N | 10 |

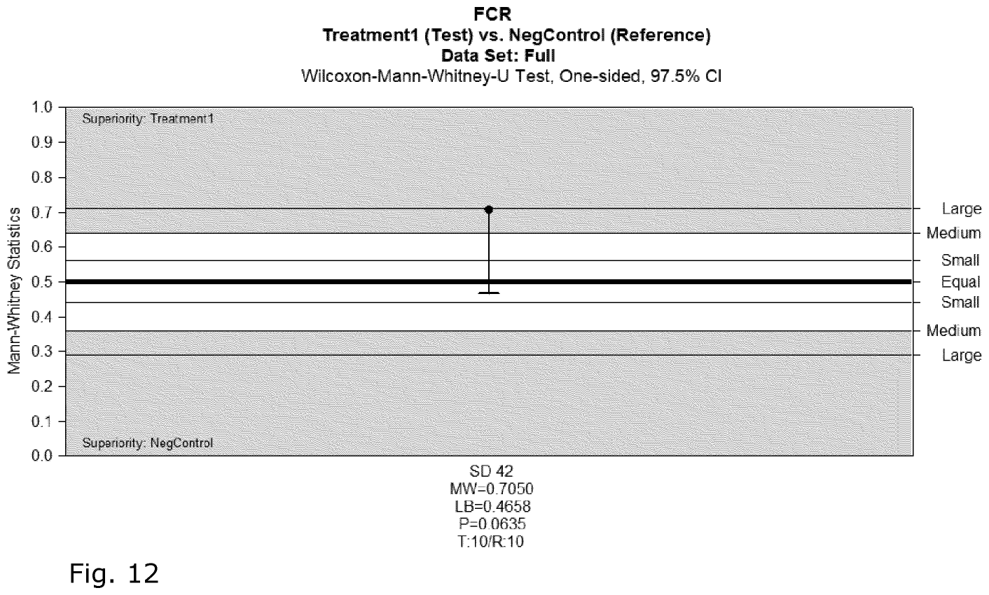

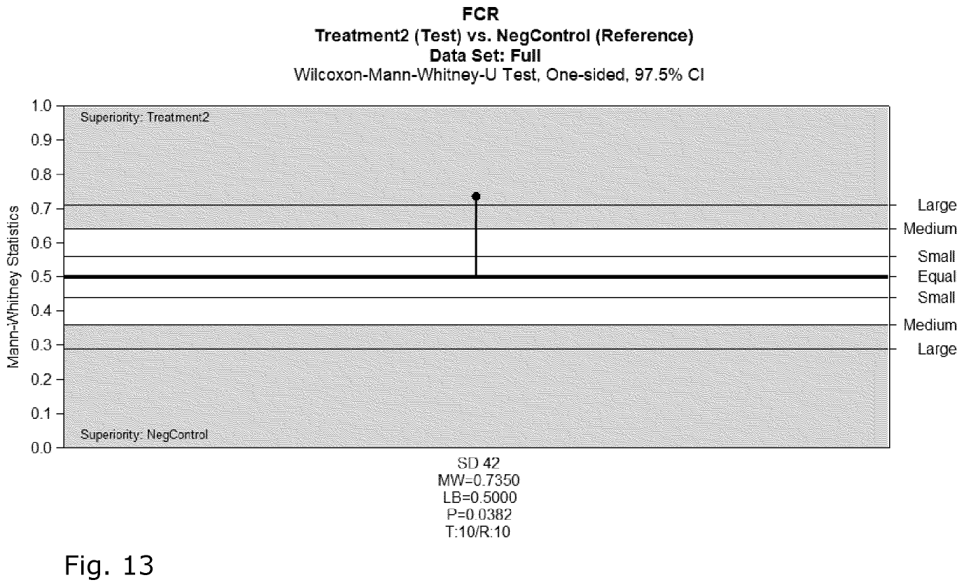

As also shown in FIGS. 12 and 13, the pigs receiving treatment 1 and treatment 2 (both including Isosteviol) had a significant decreased feed conversion ratio compared to the control group (not receiving Isosteviol) after the 42 days trial.

### Conclusion

Addition of Isosteviol to the feed, results in an increased weight gain of the pigs as well as a significantly decreased Feed conversion ratio.

Notably, the pigs were not subjected to extensive/unusual exercise during the trial. Thus, the pigs were not glycogen depleted.

1-46. (canceled)

47. A composition or food ingredient comprising isosteviol, wherein the composition or food ingredient comprises an amount of protein of at least 30 w %.

48. The composition or food ingredient according to claim 47, having an amount of isosteviol in the range of 0.1 w % to 50 w % based on the total weight of the composition or food ingredient.

49. The composition or food ingredient according to claim 47, wherein the amount of steviol glycosides make up 0 w % to 7 w % of the total weight of the composition or food ingredient.

50. The composition or food ingredient according to claim 47, wherein the amount of protein is in the range of 30 w % to 99.9 w % based on the total weight of the composition or food ingredient.

51. The composition or food ingredient according to claim 47, further comprising one or more free amino acids.

52. The composition or food ingredient according to claim 47, wherein the composition or food ingredient is in the form of grains, pellets, liquid, solid, slurry or powder.

53. A food comprising the composition or food ingredient according to claim 47, wherein the food comprises an amount of protein of at least 30 w %.

54. The food according to claim 53, wherein the food comprises a drinkable liquid.

55. The food according to claim 53, wherein the food is in a form selected from the group consisting of grains, pellets, powder, solution, suspension, dispersion, and slurry.

56. The food according to claim 53, wherein the food is selected from the group consisting of a feed, bar, sugar bar, protein bar, powder, gel, beverage, yoghurt, chewing gum, dairy product, sports drink, confectionary product, ice cream, capsule, tablet, sachet, and pouch.

57. Use of the composition or food ingredient according to claim 47 in or as a food for an animal or human.

58. The use according to claim 57, wherein the animal or human is a racing animal or athlete, subject to exhaustive exercise and the food is distributed to said racing animal or athlete before, during, and/or after the exhaustive exercise.

59. The use according to claim 57, wherein the animal is depleted in muscle glycogen.

60. The use according to claim 57, wherein the animal is not depleted in muscle glycogen.

61. Use of isosteviol or the use according to claim 57, for increasing the muscle to fat ratio of the animal to which the food is distributed compared to an animal fed with a standard food or a standard food supplemented with protein and/or carbohydrates and/or free amino acids.

62. Use of isosteviol or the use according to claim 57, for decreasing the food conversion ratio (FCR) of the animal to which the food is distributed compared to an animal fed with a standard food or a standard food supplemented with protein and/or carbohydrates and/or free amino acids.

63. Use of isosteviol or the use according to claim 57, for increasing the uptake of proteins and/or carbohydrates and/or free amino acids of the animal compared to an animal fed with a standard food or a standard food supplemented with protein and/or carbohydrates and/or free amino acids.

64. Use of isosteviol or use of the composition or food ingredient according to claim 47, for increasing the growth rate of an animal compared to an animal fed with a standard food or a standard food supplemented with protein and/or carbohydrates and/or free amino acids.

65. Use of isosteviol or the composition or food ingredient according to claim 47, for prevention and/or treatment and/or alleviation of a muscle disease/injury.

66. Use of isosteviol or the composition or food ingredient according to claim 47, for prevention and/or treatment and/or alleviation of a digestive disorder.
